(19) United States
(12) Patent Application Publication
(10) Pub. No.: US 2010/0228331 A1
(43) Pub. Date: Sep. 9, 2010

(54) IMPLANTABLE MEDICAL LEAD HAVING A BODY WITH HELICAL CABLE CONDUCTOR CONSTRUCTION AND METHOD OF MAKING SAME

(75) Inventor: Steven R. Conger, Agua Dulce, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/400,564

(22) Filed: Mar. 9, 2009

(51) Int. Cl. A61N 1/05

(52) U.S. Cl. 607/122

(57) ABSTRACT

Disclosed herein is an implantable medical lead. The lead may include a longitudinally extending body having a distal end, a proximal end, a helical core assembly extending between the distal and proximal ends, and an outer jacket about the helical core assembly. The helical core assembly may have at least one helical ridge. In some instances, the at least one helical ridge may be at least two helical ridges and the helical core may further include least two helical troughs. In some such cases, the at least two helical ridges may define the at least two helical troughs.

### FIELD OF THE INVENTION

The present invention relates to medical apparatus and methods. More specifically, the present invention relates to implantable medical leads and methods of manufacturing such leads.

### BACKGROUND OF THE INVENTION

Implantable pulse generators, such as pacemakers, defibrillators, implantable cardioverter defibrillators (“ICD”) and neurostimulators, provide electrotherapy via implantable medical leads to nerves, such as those nerves found in cardiac tissue, the spinal column, the brain, etc. Electrotherapy is provided in the form of electrical signals, which are generated in the pulse generator and travel via the lead's conductors to the electrotherapy treatment site.

Lead conductors are typically in the form of flexible single wires or multi-filar cables. These lead conductors may be individually electrically insulated with their own dedicated insulation jackets or may be without a dedicated insulation jacket, instead having to rely on the concentric insulation layers of the lead body.

A lead conductor typically has one of two configurations for its routing through a lead body, namely, a helical coil configuration or a straight configuration. As can be understood from FIG. 1, which is a longitudinal cross-section of a segment of a common lead body 1, a helical coil conductor 2 has a small helical pitch, resulting in adjacent coils 3′, 3″ of the helical coil conductor 2 abutting each other or nearly abutting to form a tightly wound helical coil 2. As is the case in FIG. 1, such helical coil conductors 2 often form the core of the lead body 1 and define a central lumen 4 through which a stylet or guidewire may be extended when implanting the lead. Multiple helical coil conductors 2 may exist in a single lead body, the coil conductors being concentrically arranged. Due to their small pitches and being tightly wound, helical coil conductors 2 require a substantial length of conductor material to extend the length of the lead body 1. This extreme length of conductor material increases the cost of implantable medical leads. Also, a tightly wound helical coil conductor 2 may provide substantial stiffness to the lead body 1, increasing the likelihood of the lead penetrating heart tissue. The lead body stiffness may increase substantially for each additional helical coil conductors 2 concentrically employed in the lead body 1. Also, the diameter of the lead body may increase with each additional conductor.

As can be understood from FIG. 1, to provide the benefit of a central lumen 4 and keep the cost and lead body stiffness to a minimum, the lead body 1 may employ a “helical coil” conductor 2 for one of its conductors, thereby forming the core and central lumen 4 of the lead body 1. The other lead conductors 5 employed by the lead body 1 may then be conductors 5 having a straight route configuration.

As can be understood from FIG. 2, which is a longitudinal cross-section of a segment of another common lead body 1, to eliminate the cost and lead body stiffness associated with helical coil conductors 2, the lead body 1 may have a central lumen 4 formed of a polymer sheath 6 and the conductors 5 extending through the lead body 1 may all be conductors 5 having a straight route configuration.

As indicated in FIGS. 1 and 2, conductors 5 having a straight route configuration extend in a straight route through the lead body 1. Such “straight-routed” conductors 5 are typically spaced apart from, or located off of, the lead body's neutral axis of flexure. The combination of being “straight-routed” and offset from the natural axis of flexure subjects the straight-routed conductors 5 to substantial normal strains in tension and compression when the lead body 1 is deflected. The magnitude of the strains can be significant even when the lead body 1 is configured such that its straight-routed conductors 5 are located in lumens 7 so as to be able to displace within the lead body 1 at least a small amount to relieve via displacement the body deflection generated stresses in the straight-routed conductors 5. However, the magnitude of the strains is especially great when the straight-routed conductors 5 are “potted” in lead body materials or otherwise constrained from displacing within the lead body 1. The strains can result in premature failure of the straight-routed conductors 5.

New lead technologies and treatment programs make it desirable to place electronic lead components along the length of the lead body 1. For example, as indicated in FIG. 3, which is an isometric view of a segment of a proposed lead body 1, multiple fragile electronic chips 8 may be located along the lengths of the straight-routed conductors 5. The placement of such electronic chips 8 necessitates multiple closely spaced couplings 9 of the straight-routed conductors 5 with the terminals of the electronic chips 8. Such close spaced couplings 9 with straight-routed conductors 5 substantially reduce the ability of the straight-routed conductors 5 to displace and conform to displacement of the lead body 1, potentially resulting in rapid failure of the straight-routed conductors 5. Also, the straight-routed conductors 5 result in substantial strain in the couplings 9, causing rapid failure of the couplings 9 as well.

New lead technologies and treatment programs also make it desirable to deliver leads to non-traditional implantations sites. For example, implantable leads may be delivered sub-xyphoid to an intrapericardial implantation site. As a result, such leads will be subjected to tunneling, hard contact with bone, and various shear and buckling loads associated with torso movement, increasing the likelihood of early failure for straight-routed conductors.

Lead construction for leads employing straight-routed conductors 5 is expensive due to the need for costly multi-lumen tubing extrusions and labor-intensive and operator dependent “stringing” of conductors.

There is a need in the art for a lead having a conductor configuration that provides improved resistance to strain induced conductor failure, reduced lead body stiffness and reduced manufacturing costs. There is also a need in the art for a method of manufacturing a lead having such a conductor configuration.

### BRIEF SUMMARY OF THE INVENTION

Disclosed herein is an implantable medical lead. In one embodiment, the lead may include a longitudinally extending body having a distal end, a proximal end, a helical core assembly extending between the distal and proximal ends, and an outer jacket about the helical core assembly. The helical core assembly may have at least one helical ridge. In one embodiment, the at least one helical ridge may be at least two helical ridges and the helical core may further include least two helical troughs. The at least two helical ridges may define the at least two helical troughs.

Disclosed herein is a method of assembling a medical lead. In one embodiment, the method includes: providing a longitudinally extending helical core assembly including at least one helical ridge; and providing an outer jacket about the helical core assembly. In one embodiment, the at least one helical ridge may be at least two helical ridges and the helical core may further include least two helical troughs. The at least two helical ridges may define the at least two helical troughs.

Disclosed herein is an implantable medical lead. In one embodiment, the lead includes a longitudinally extending body including a distal end, a proximal end, and a helical core assembly extending between the distal and proximal ends. The helical core assembly includes an inner tube liner and a helically-routed conductor having a wind pitch of between approximately 0.05″ and approximately 0.3″ and routed about the inner tube liner. In one embodiment, an infill polymer material extends around the helical core assembly to cause the helical core assembly to be generally isodiametric. In other embodiments, a conformal jacket extends around the inner tube liner and conductor in a conforming fashion such that the helical core assembly has a ridge and a trough.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a longitudinal cross-section of a segment of a common lead body employing a helical coil conductor defining a core and central lumen of the lead body, the lead also employing straight-routed conductors.

FIG. 2 is a longitudinal cross-section of a segment of another common lead body, wherein the lead body may have a central lumen formed of a polymer sheath and the conductors extending through the lead body are all straight-routed conductors.

FIG. 3 is an isometric view of a segment of a proposed lead body, wherein multiple fragile electronic chips may be located along the lengths of straight-routed conductors.

FIG. 4 is an isometric view of an implantable medical lead and a pulse generator for connection thereto.

FIG. 5A is an isometric view of a longitudinal segment of the lead body with the outer jacket of the lead body mostly hidden to reveal a helical core assembly of the lead body.

FIG. 5B is a longitudinal side view of the lead body of FIG. 5A with the outer jacket shown in phantom lines to reveal the helical core assembly.

FIG. 5C is a transverse cross-section of the lead body as taken along section line 5C-5C in FIG. 5B.

FIG. 5D is an isometric diagrammatic view of the inner liner and the helically-routed conductors of the helical core assembly, wherein the helically-routed conductors helically extend along the inner liner.

FIGS. 5E-5H are views similar to that depicted in FIG. 5A, except of alternative embodiments.

FIG. 6A is the same isometric view as FIG. 5A illustrating the same lead body with the same helical core assembly, except with outer conductors routed through one of the two troughs of the helical core assembly.

FIG. 6B is a longitudinal side view of the lead body of FIG. 6A with the outer jacket shown in phantom lines to reveal the helical core assembly.

FIG. 6C is a transverse cross-section of the lead body as taken along section line 6C-6C in FIG. 6B.

FIG. 7A is the same isometric view as FIG. 5A illustrating the same lead body with the same helical core assembly, except with outer conductors routed through both of the two troughs of the helical core assembly.

FIG. 7B is a longitudinal side view of the lead body of FIG. 7A with the outer jacket shown in phantom lines to reveal the helical core assembly.

FIG. 7C is a transverse cross-section of the lead body as taken along section line 7C-7C in FIG. 7B.

FIG. 8A is the same isometric view as FIG. 6A illustrating the same lead body with the same helical core assembly, except with a mechanical element extending through a helical trough for biasing the lead body into a desired shape.

FIG. 8B is a longitudinal side view of the lead body of FIG. 8A with the outer jacket shown in phantom lines to reveal the helical core assembly.

FIG. 8C is a transverse cross-section of the lead body as taken along section line 8C-8C in FIG. 8B.

FIG. 9 is a diagram illustrating a process of manufacturing a lead body employing the helical core assembly disclosed herein.

FIGS. 10A and 10B are views similar to that depicted in FIG. 5C, except of another embodiment.

### DETAILED DESCRIPTION

An implantable medical lead 10 is disclosed herein. In one embodiment, the implantable medical lead 10 includes a helical core assembly 110 that forms the central core of the lead body 50. The helical core assembly 110 may include one or more “helically-routed” conductors 85, 90 that extend through the helical core assembly 110 in a helical arrangement that has a helical pitch that is relatively large as compared the above-discussed “helical coil” conductors 2.

Unlike the above-discussed helical coil conductors 2, in some embodiments, the helically-routed conductors 85, 90 of the lead 10 may have a large helical pitch. For example, the pitch of the helically-routed conductors 85, 90 may be so great that the overall length of a helically-routed conductor 85, 90 is not substantially greater than the overall length of straight-routed conductors 5 for the same lead body 50. The helical configuration of the conductors 85, 90 serves to effectively decouple the conductors 85, 90 from the normal strains of the lead body 50 in bending, even if the conductors 85, 90 are potted in the material of the lead body's jacket 105. Also, the helical configuration may provide rolling, deflection, and feel that is more consistent during implantation than the rolling, deflection and feel provided by lead bodies with straight routed conductors.

In some embodiments, the pitch may be small, medium or large such that the overall length of the conductors 85, 95 exceeds the overall length of straight-routed conductors to a greater or lesser extent. Also, in some embodiments, the pitch may vary for a conductor as it extends along the lead body.

In one embodiment where the helically-routed conductors 85, 90 are routed along the longitudinal axis of the lead body radially spaced apart from each other, the coils 85′, 90′ of the helically-routed conductors 85, 90 do not abut adjacent coils 85″, 90″. In one embodiment where the helically-routed conductors 85, 90 are routed along the longitudinal axis of the lead body radially adjacent to each other, the coils 85′, 90′ of the helically-routed conductors 85, 90 may abut adjacent coils 85″, 90″.

In one embodiment, the helical core assembly 110 may be provided in a preassembled state to include a removable core wire 175, a liner tube 120 surrounding the core wire 175, a pair of helically wound conductors 85, 90 routed helically about the tube 120, and a thin conformal jacket 125 extending about the conductors 85, 90 and tube 120. In such a preassembled state, the helical core assembly 110 may act as a “universal platform” 110 and foundation for constructing a wide variety of lead types and substantially reducing the complexity and costs associated with manufacturing leads 50.

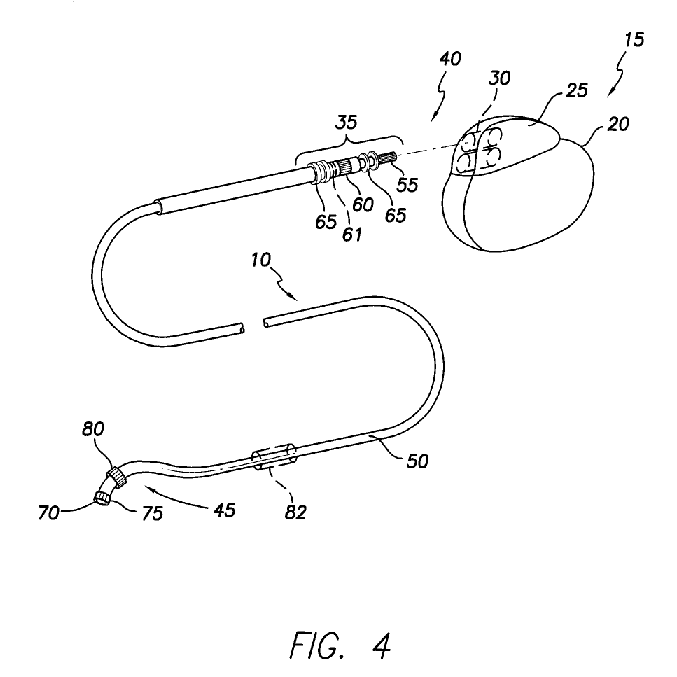

For a general discussion of an embodiment of a lead 10 employing the helically-routed conductor configuration, reference is made to FIG. 4, which is an isometric view of the implantable medical lead 10 and a pulse generator 15 for connection thereto. The pulse generator 15 may be a pacemaker, defibrillator, ICD or neurostimulator. As indicated in FIG. 4, the pulse generator 15 may include a can 20, which may house the electrical components of the pulse generator 15, and a header 25. The header may be mounted on the can 20 and may be configured to receive a lead connector end 35 in a lead receiving receptacle 30.

As shown in FIG. 4, in one embodiment, the lead 10 may include a proximal end 40, a distal end 45 and a tubular body 50 extending between the proximal and distal ends. In some embodiments, the lead may be a 6 French, model 1688T lead, as manufactured by St. Jude Medical of St. Paul, Minn. In other embodiments, the lead may be a 6 French model 1346T lead, as manufactured by St. Jude Medical of St. Paul, Minn. In other embodiments, the lead 10 may be of other sizes and models.

As indicated in FIG. 4, the proximal end 40 may include a lead connector end 35 including a pin contact 55, a first ring contact 60, a second ring contact 61, which is optional, and sets of spaced-apart radially projecting seals 65. In some embodiments, the lead connector end 35 may include the same or different seals and may include a greater or lesser number of contacts. The lead connector end 35 may be received in a lead receiving receptacle 30 of the pulse generator 15 such that the seals 65 prevent the ingress of bodily fluids into the respective receptacle 30 and the contacts 55, 60, 61 electrically contact corresponding electrical terminals within the respective receptacle 30.

As illustrated in FIG. 4, in one embodiment, the lead distal end 45 may include a distal tip 70, a tip electrode 75 and a ring electrode 80. In some embodiments, the lead distal end 45 may include a helical anchor that is extendable from within the distal tip 70 for active fixation and may or may not act as an electrode. In other embodiments, the lead distal end 45 may include features or a configuration that facilitates passive fixation.

As shown in FIG. 4, in some embodiments, the distal end 45 may include a defibrillation coil 82 about the outer circumference of the lead body 50. The defibrillation coil 82 may be located proximal of the ring electrode 70.

The tip electrode 75 may form the distal tip 70 of the lead body 50. The ring electrode 80 may extend about the outer circumference of the lead body 50, proximal of the distal tip 70. In other embodiments, the distal end 45 may include a greater or lesser number of electrodes 75, 80 in different or similar configurations.

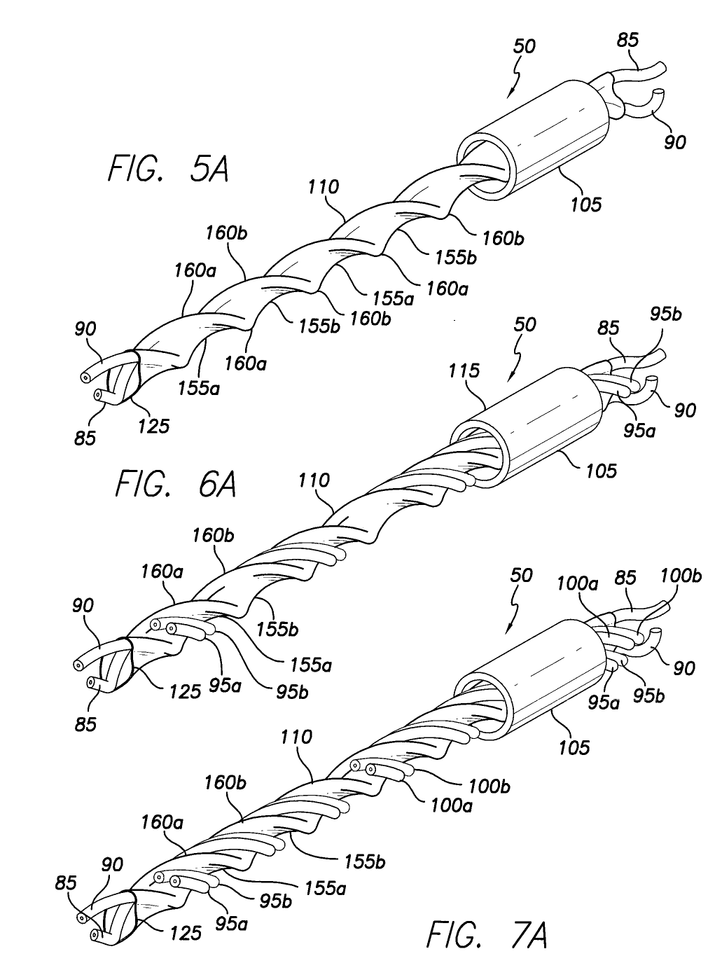

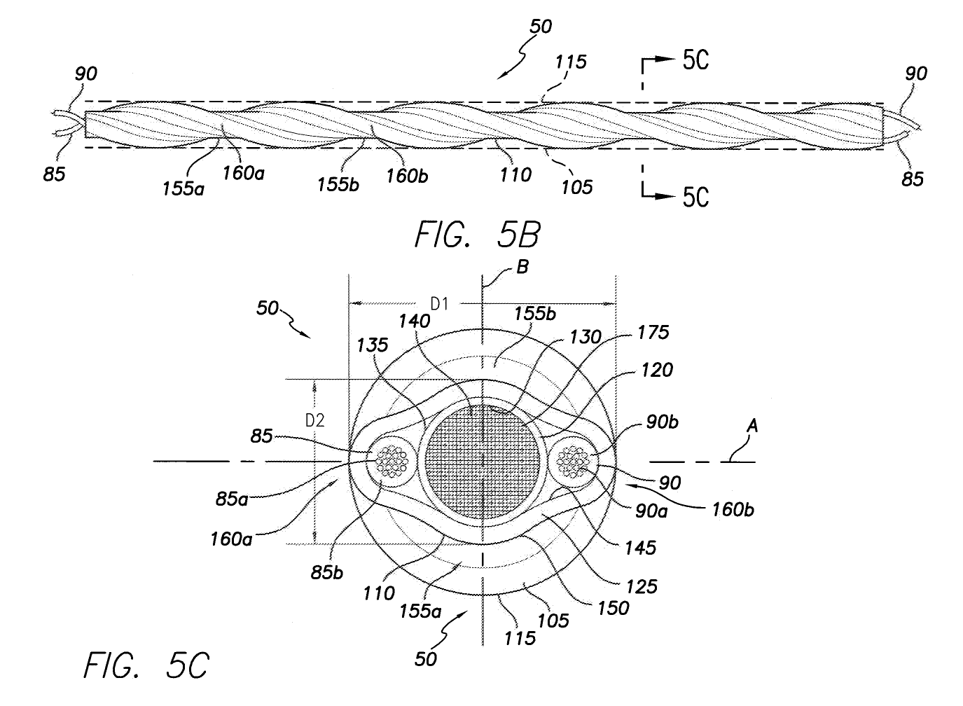

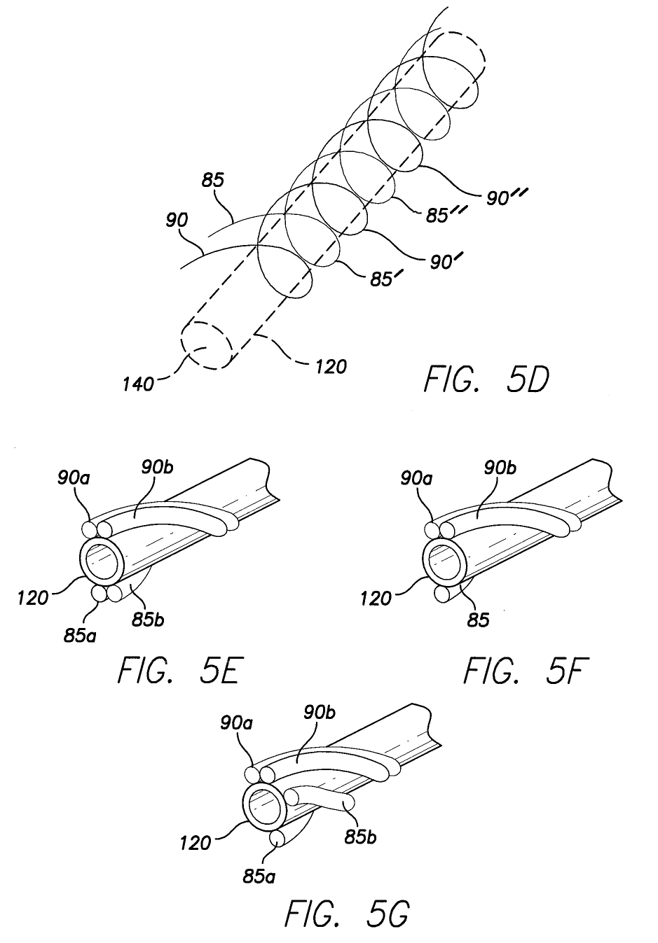

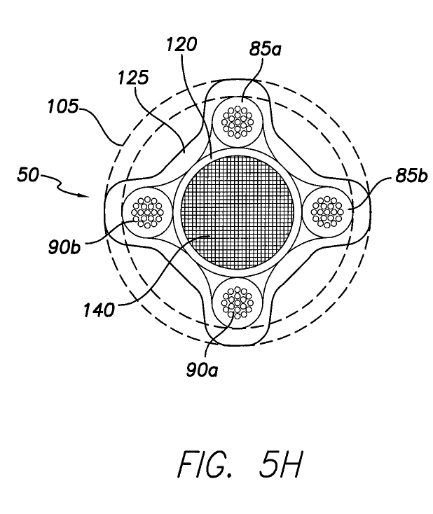

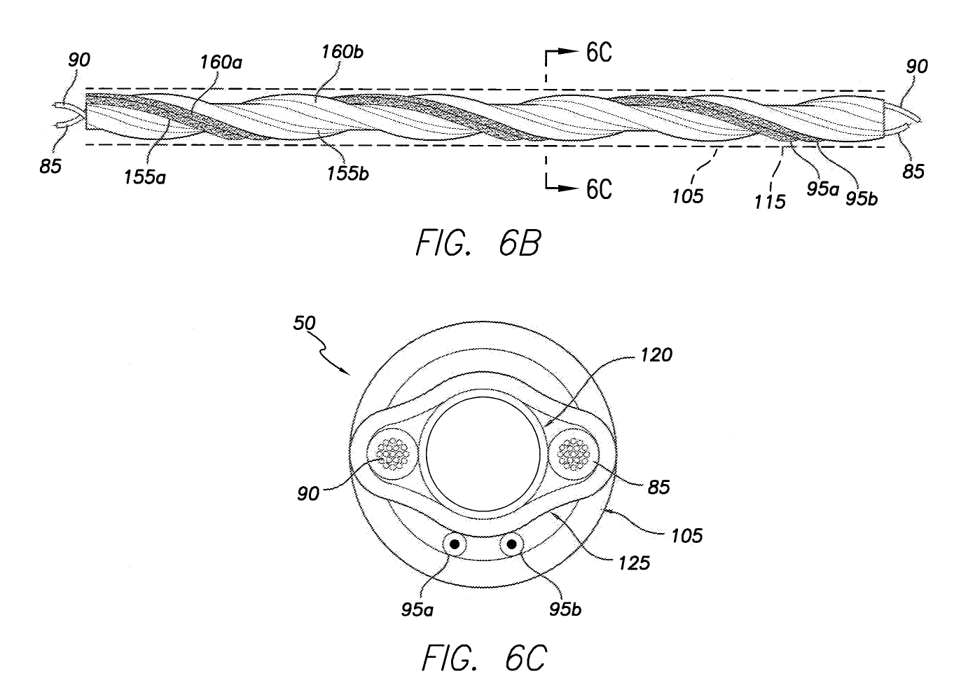

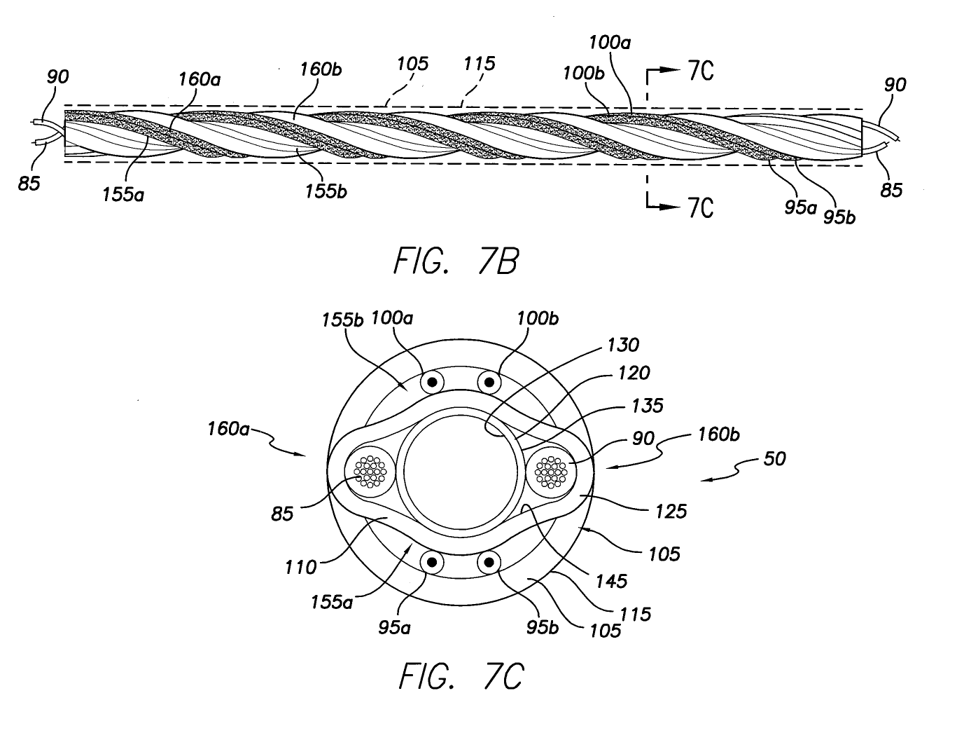

In one embodiment, the tip electrode 75 may be in electrical communication with the pin contact 55 via a first electrical conductor 85 (see FIGS. 5A-5C) and the ring electrode 80 may be in electrical communication with the first ring contact 60 via a second electrical conductor 90 (see FIGS. 5A-5C). In some embodiments, the defibrillation coil 82 may be in electrical communication with the second ring contact 61 via a third electrical conductor or pair of conductors 95 (see FIGS. 6A-6C). In yet other embodiments, other lead components (e.g., additional ring electrodes, various types of sensors, etc.) mounted on the lead body distal region 45 or other locations on the lead body 50 may be in electrical communication with a third ring contact (not shown) similar to the second ring contact 61 via a fourth electrical conductor or pair of electrical conductors 100 (see FIGS. 7A-7C). Of course, if needed, electrical conductors in addition to the four conductors 85, 90, 95, 100 already mentioned may be routed through the lead body in a manner similar to that depicted in FIGS. 5A-7C. Depending on the embodiment, any one or more of the conductors 85, 90, 95, 100 may be a multi-strand or filar cable, as indicated with respect to conductors 85, 90 in FIGS. 5C, 6C and 7C, or a single solid wire conductor run singly or grouped, for example in a pair, as indicated with respect to conductors 95, 100 in FIGS. 6C and 7C.

For a detailed discussion regarding a lead body 50 employing the “helically-routed” conductor configuration disclosed herein, reference is made to FIGS. 5A-5D. FIG. 5A is an isometric view of a longitudinal segment of the lead body 50 with the outer jacket 105 of the lead body 50 mostly hidden to reveal a helical core assembly 110 of the lead body 50. FIG. 5B is a longitudinal side view of the lead body 50 of FIG. 5A with the outer jacket 105 shown in phantom lines to reveal the helical core assembly 110. FIG. 5C is a transverse cross-section of the lead body 50 as taken along section line 5C-5C in FIG. 5B. FIG. 5D is an isometric diagrammatic view of the inner liner 120 and the helically-routed conductors 85, 90 of the helical core assembly 110, wherein the helically-routed conductors 85, 90 helically extend along the inner liner 120.

As indicated in FIGS. 5A-5C, in one embodiment, the helical core assembly 110 forms a central or core portion 110 of the lead body 50 and is enclosed by the outer jacket 105, which forms the outer circumferential surface 115 of the lead body 50. The outer jacket 105 may be formed of silicone rubber, silicone rubber—polyurethane—copolymer (“SPC”), polyurethane, etc.

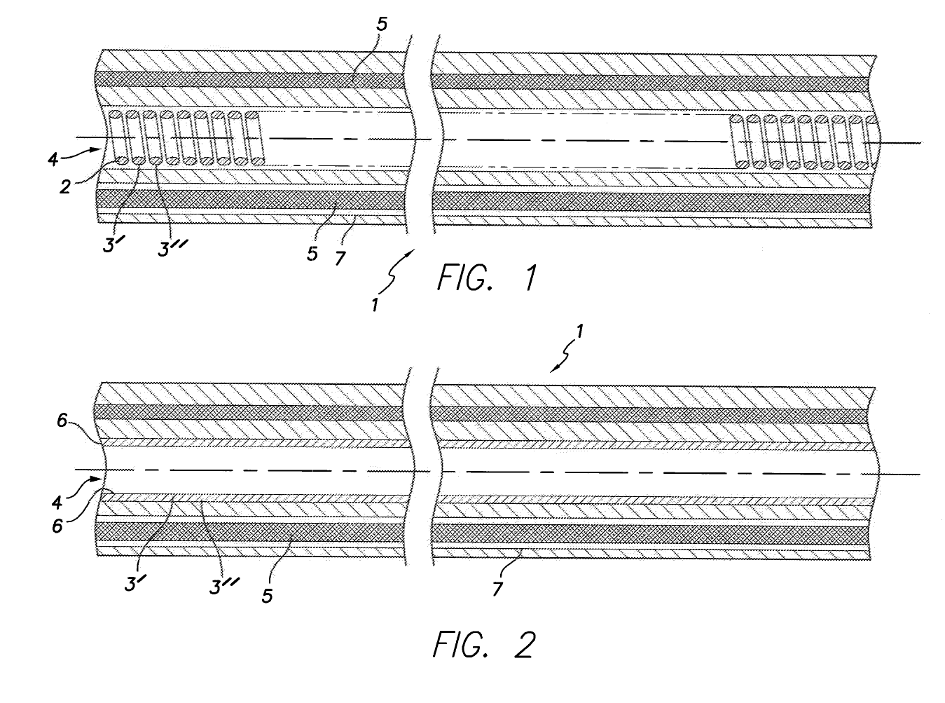

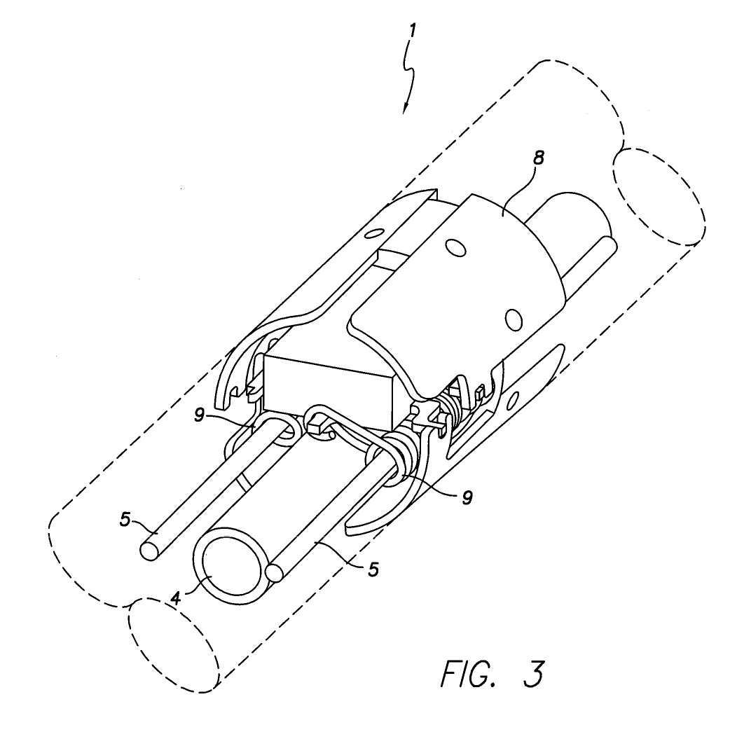

As illustrated in FIG. 5C, in one embodiment, the helical core assembly 110 includes an inner liner 120, a pair of conductors 85, 90, and a core jacket 125. The inner liner 120 includes inner and outer circumferential surfaces 130, 135. The inner circumferential surface 130 of the inner liner 120 may define a lumen 140, which may serve as the central lumen of the lead body 50 and through which guidewires and stylets may be extended during the implantation of the lead 10. In one embodiment, the inner liner 120 may be formed of a polymer material such as ethylene tetrafluoroethylene (“ETFE”), polytetrafluoroethylene (“PTFE”), etc. In other embodiments, the inner liner 120 may be formed of a helical coil conductor 2 similar to that discussed above with respect to FIG. 1.

As indicated in FIG. 5C, in one embodiment, two conductors 85, 90 are located outside the inner liner 120 adjacent to the outer circumferential surface 135 of the inner liner 120. The two conductors 85, 90 may be evenly radially spaced from each other about the outer circumferential surface 135 of the inner liner 120. The conductors 85, 90 have electrically conductive cores 85a, 90a and may or may not have electrical insulation jackets 85b, 90b of their own. Where the conductors 85, 90 have insulation jackets 85b, 90b, the insulation jackets 85b, 90b may be formed of a polymer material such as ETFE, PTFE, etc. The electrically conductive cores 85a, 90a may be multi-wire or multi-filar cores or solid single wire cores.

As depicted in FIG. 5C, the helical core assembly 110 may have two conductors 85, 90 that are evenly radially spaced apart from each other about the inner liner 120. However, in other embodiments, the conductors 85 may have other arrangements. For example, as shown in FIG. 5E, which is an isometric view similar to FIG. 5A, the helical core assembly 110 may include greater than or less than two conductors 85, 90, and the conductors 85, 90 may be routed in groups (e.g., pairs, etc.) of conductors 85a, 85b and 90a, 90b such that the conductors are not radially spaced apart. More specifically, the coils of the helically routed conductors 85a, 85b and 90a, 90b may actually contact each other despite having a pitch that results in an overall length that is not substantially greater than a straight-routed conductor.

As illustrated in FIG. 5F, which is an isometric view similar to FIG. 5A, some of the conductors 90a, 90b may be routed in groups while other conductors 85 are not grouped. Also, as indicated in FIG. 5G, which is an isometric view similar to FIG. 5A, the conductors 85a, 85b and 90a, 90b may or may not be evenly radially spaced apart from each other about the inner liner whether routed in groups or individually. Thus, the helical core assembly 110 may have any number of wiring configurations that employ the helically-routed conductor concepts disclosed herein. As indicated in FIG. 5H, which is an isometric view similar to FIG. 5A, the lead may any number of conductors, including a single conductor, two conductors, three conductors, four conductors, etc. Thus, the lead may have sufficient conductors 85, 90 to allow a lead 10 to be single polar, bi-polar tri-polar, quad-polar, or possibly more poles.

As can be understood from FIGS. 5A, 5B and 5D, the conductors 85, 90 longitudinally extend along the outer circumferential surface 135 of the inner liner 120 in a helical wind. In one embodiment, the “helically-routed” conductors 85, 90 extend through the helical core assembly 110 in a helical arrangement that has a helical pitch that is relatively large as compared the above-discussed “helical coil” conductors 2.

As illustrated in FIG. 5D, in one embodiment, unlike the above-discussed helical coil conductors 2 and due to the large helical pitch of the helically-routed conductors 85, 90, the adjacent coils 85′, 85″ of a specific conductor 85 do not abut against each other. Also, in some embodiments where the multiple conductors 85, 90 are radially spaced apart from each other about the outer circumferential surface 135 of the inner liner 120 as indicated in FIG. 5C, the coils 85′, 85″ of a first conductor 85 will not abut against the corresponding adjacent coils 90′, 90″ of a second conductor 90 as shown in FIG. 5D.

As best understood from FIGS. 5A and 5B, in one embodiment, the pitch of the helically-routed conductors 85, 90 is so great that the overall length of a helically-routed conductor 85, 90 if placed in a straight non-helical condition is not substantially greater than the overall length of a straight-routed conductor 5 for the same length of lead body 50. In one embodiment, the pitch of the helically-routed conductors 85, 90 is between approximately 0.05″ and approximately 0.3″.

As shown in FIG. 5C, the core jacket 125 includes an inner surface 145 and an outer surface 150. The core jacket 125 extends about the conductors 85, 90 and the inner liner 120, thereby enclosing the inner liner 120 and the conductors 85, 90 within the core jacket 125.

As depicted in FIG. 5C, the core jacket 125 may snuggly fit about the inner liner 120 and the conductors 85, 90 such that the inner surface 145 of the core jacket 125 extends along and generally conforms to portions of the outer circumferential surface 135 of the inner liner 120 and the outer surfaces of the conductors 85, 90 (e.g., the outer surfaces of the conductor insulation 85b, 90b, where present). Where there are two conductors 85, 90, the resulting transverse cross-section of the helical core assembly 110 may have a first diameter D1, which is aligned with a first axis A extending through the center points of the conductors 85, 90 and lumen 140, that is substantially longer than a second diameter D2, which aligned with a second axis B that is generally perpendicular to the first axis A.

As shown in FIGS. 5A and 5B, on account of the helical routing of the conductors 85, 90 about the inner liner 120 and the general conforming of the core jacket 125, the outer surface 150 of the core jacket 125 is helical, defining helically extending troughs 155a, 155b separated by helically extending ridges 160a, 160b. Where the helical core assembly 110 includes two helically-routed conductors 85, 90 and the core jacket 125 generally conforms to the conductors 85, 90 and inner liner 120, the outer surface 150 of the core jacket 125 may have a pair of troughs 155a, 155b and a pair of ridges 160a, 160b. Where the helical core assembly 110 includes one, three, four, five and so forth helically-routed conductors and the core jacket 125 generally conforms to the conductors and inner liner 120, the outer surface 150 of the core jacket 125 may have respectively one, three, four, five and so forth troughs and one, three, four, five and so forth ridges.

As can be understood from FIGS. 5A-5C, the location and routing of each helically extending ridge 160a, 160b corresponds and generally matches the location and routing of a specific helically-routed conductor 85, 90. The location and routing of each helically extending trough 155a, 155b corresponds and generally matches the location of a space centered between a pair of helically-routed conductors 85, 90.

As indicated in FIG. 5C, in one embodiment, the helical core assembly 110 is encased or imbedded in the material of the outer jacket 105 of the lead body 50, the outer circumferential surface 115 of the outer jacket 105 forming the outer circumferential surface 115 of the lead body 50. As indicated in FIG. 5C, the outer jacket 105 may be such that it in-fills the voids between the lead body outer circumferential surface 115 and the core jacket outer surface 150 in the vicinity of the troughs 155a, 155b. The result is a lead body 50 with an outer circumferential surface 115 having a generally circular shape in transverse cross-section and generally uniform diameter along its length, despite the helical core assembly 110 having a transverse cross-section that is semi-elliptical.

As indicated in FIGS. 6A-6C, which are the same respective views as FIGS. 5A-5C, additional or outer conductors 95 may be routed through one of the two troughs 155a of the helical core assembly 110. The outer conductors 95 may be a single conductor, a pair of conductors 95a, 95b, or more conductors helically routed along a specific helical trough 155a. The outer conductors 95a, 95b may be encased or imbedded in the material of the outer jacket 105.

As indicated in FIGS. 7A-7C, which are the same respective views as FIGS. 6A-6C, in addition to the outer conductors 95 routed through the first trough 155a, yet more additional or outer conductors 100 may be routed through the other trough 155b of the two troughs 155a of the helical core assembly 110. The yet more outer conductors 100 may be a single conductor, a pair of conductors 100a, 100b, or more conductors helically routed along a specific helical trough 155b. The outer conductors 95a, 95b, 100a, 100b may be encased or imbedded in the material of the outer jacket 105.

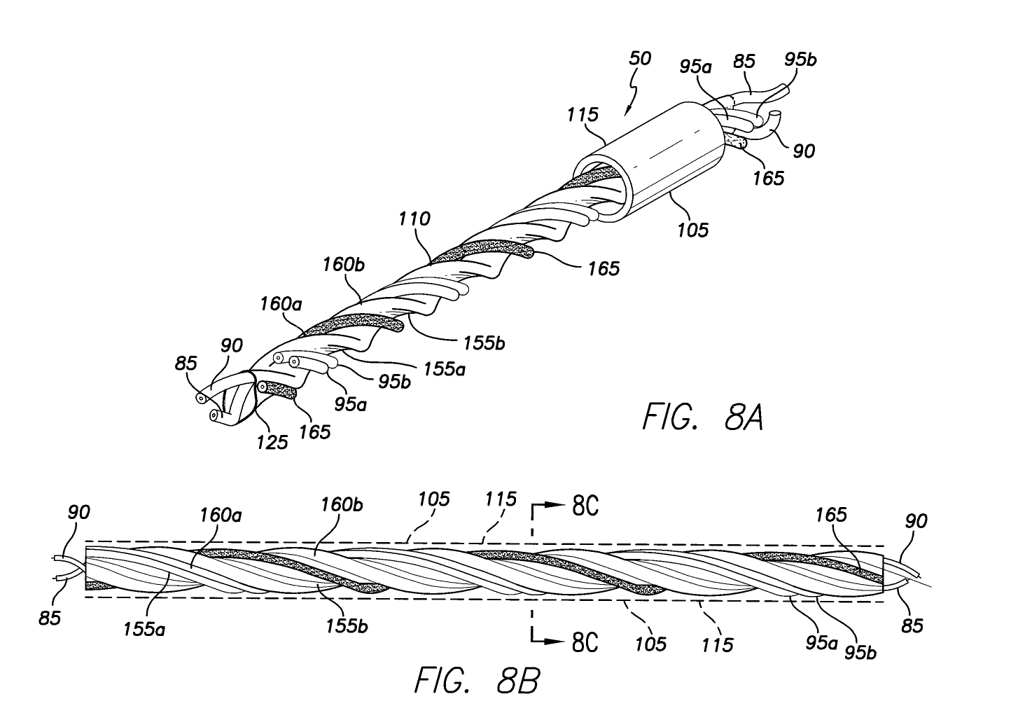

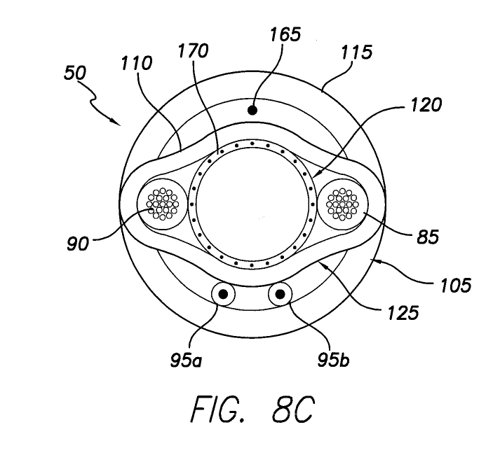

As indicated in FIGS. 8A-8C, which are the same respective views as FIGS. 6A-6C, in addition to the outer conductors 95 routed through the first trough 155a, mechanical elements 165 (e.g., helical spring coils, etc.) may be provided as part of the helical core assembly 110 to affect the shape reinforcement or fixation function of the lead body 50. For example, a mechanical element 165 may have a helical configuration and be routed through a trough 155b that is free of outer conductors 95, as illustrated in FIGS. 8A-8C. Alternatively, the mechanical element 165 may occupy the same trough 155a as the outer conductors 95. In one embodiment, there may be multiple mechanical elements 165, which may be located in a single trough 155 or both troughs 155a, 155b. The mechanical element 165 may be encased or imbedded in the material of the outer jacket 105. In one embodiment, the mechanical element 165 is formed of stainless steel, MP35N, Nitinol, etc.

As indicated in FIG. 8C, in one embodiment, mechanical behavior modifiers 170 (e.g., a tubular braid reinforcement) may be incorporated into the inner liner 120 to promote lead torqueability, etc.

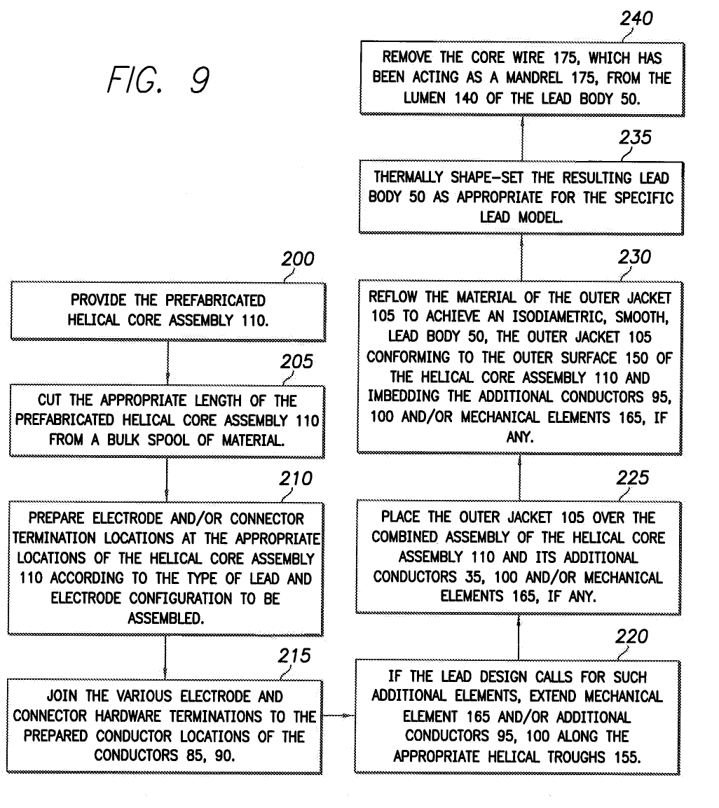

For a discussion of a method of assembling a lead body 50 employing a helical core assembly 110 as described in any of FIGS. 5A-8C, reference is made to FIG. 9, which is a manufacturing process diagram for the assembly of lead bodies 50 employing the above-described helical core assembly 110. In one embodiment, the helical core assembly 110 may be provided in a preassembled state to include a removable core wire 175, a liner tube 120 surrounding the core wire 175, a pair of helically wound conductors 85, 90 routed helically about the tube 120, and a thin conformal jacket 125 extending about the conductors 85, 90 and tube 120 [(block 200) of FIG. 9]. For example, the helical core assembly 110 may be prefabricated and procured on bulk spools. The appropriate length of prefabricated helical core assembly 110 is first cut from a bulk spool of material [(block 205) of FIG. 9]. Electrode and/or connector termination locations are prepared at the appropriate locations of the helical core assembly 110 according to the type of lead and electrode configuration to be assembled [(block 210) of FIG. 9]. For example, laser ablation may be used to remove the various layers of the helical core assembly 110 covering the electrically conductive aspects 85a, 90a of the conductors 85, 90.

Crimping, welding, brazing, soldering or electrically conductive epoxy are used to join the various electrode and connector hardware terminations to the prepared conductor locations of the conductors 85, 90 [(block 215) of FIG. 9]. If the lead design calls for such additional elements, mechanical elements 165 and/or additional conductors 95, 100 may be extended along the appropriate helical troughs 155 [(block 220) of FIG. 9]. For example, if the lead 10 were a tri-polar or quad-polar application, additional conductors 95, 100 may be routed along the troughs 155. Similarly, if the lead 10 is to be configured for passive fixation and relies on a mechanical element 165 to accomplish this objective, then the mechanical element 165 may be routed along a trough 155. The outer jacket 105 is placed over the combined assembly of the helical core assembly 110 and its additional conductors 35, 100 and/or mechanical element 165, if any [(block 225) of FIG. 9]. Depending on the embodiment, the outer jacket 105 may be silicone rubber, SPC, polyurethane, etc. in the form of a split tube, helical ribbon, tape wrap, etc. The material of the outer jacket 105 is then reflowed to achieve an isodiametric, smooth, lead body 50, the outer jacket 105 conforming to the outer surface 150 of the helical core assembly 110 and imbedding the additional conductors 95, 100 and/or mechanical elements 165, if any [(block 230) of FIG. 9]. The resulting lead body 50 is then thermally shape-set as appropriate for the specific lead model [(block 235) of FIG. 9]. The core wire 175, which has been acting as a mandrel 175, may be removed from the lumen 140 of the lead body 50 [(block 240) of FIG. 9]. Any additional components that could not be installed, for example, o-ring seals, steroid plugs, suture sleeves, etc., may then be installed [(block 245) of FIG. 9].

As can be understood from FIG. 5C and the preceding discussion regarding FIG. 9, the removable core wire 175 contained within the as-received pre-assembled helical core assembly 110 may be exploited as a build mandrel 175 or build wire 175. The core wire 175 may provide support to the helical core assembly 110 during handling in manufacturing. More importantly, the core wire 175 may be pulled tightly in assembly jigs and fixtures, providing stable, straight, and precisely positionable helical core assemblies 110 required for modular automated manufacturing processes. The core wire 175 is easily withdrawn from the lead body 50 or, more specifically, the helical core assembly 110, whenever required.

The above-described manufacturing approach to lead construction and assembly eliminates costly multilumen tubing extrusions and labor-intensive and operator dependent “stringing” of cable conductors. The helical core assembly 110 and the methods for its assembly into a lead body 50 are consistent with modern, highly tooled, streamlined manufacturing techniques, which have been all but impossible to employ with lead configurations known in the art.

In some embodiments, the above-described method of manufacture is highly efficient at least in part due to the manufacturing efficiency provided by the helical core assembly 110, which may act as a preassembled core 110 for the assembly of the lead body 50. Also, the core assembly 110 provides a common “universal platform” 110 and foundation for constructing a wide variety of lead types such as, for example, RA leads, RV leads, LV Brady leads, RV Tachy leads, and Intrapericardial leads. The expandable nature of the platform 110 facilitates its universality element, wherein the common core assembly 110 and manufacturing technology can be employed to manufacture a variety of different lead types.

Prototype lead bodies 50 built employing the helical core assembly 110 disclosed herein were tested and proven to have superior flex fatigue and tensile strength properties, as compared to leads having conductor configurations known in the art. For example, prototype lead bodies 50 with “helically-routed” conductors 85, 90 encased in solid SPC and having the helical core assembly 110 disclosed herein have undergone CENELEC lead body testing, logging over 90 times the CENELEC standard without failure. The Helical core assembly 110 provides a robust and durable platform offering superior flex durability and superior flexibility. In one embodiment, the construction of the helical core assembly 110 behaves as a structural unit in and of itself. The cable conductors 85, 90 are well anchored within the lead body 50, but are flexible and stress-relieved due in part to their unique helical routing geometry and the overall configuration of the helical core assembly 110.

Because the conductors 85, 90 are helically routed, they become effectively decoupled from the normal strains of the lead body 50 in bending. Even when potted in solid silicone rubber or SPC, the helically-routed conductors 85, 90 experience a stress state that provides flexural durability superior to any other known lead design in existence. Additionally, the co-helical arrangement of the conductors 85, 90 may provide favorable MRI response characteristics.

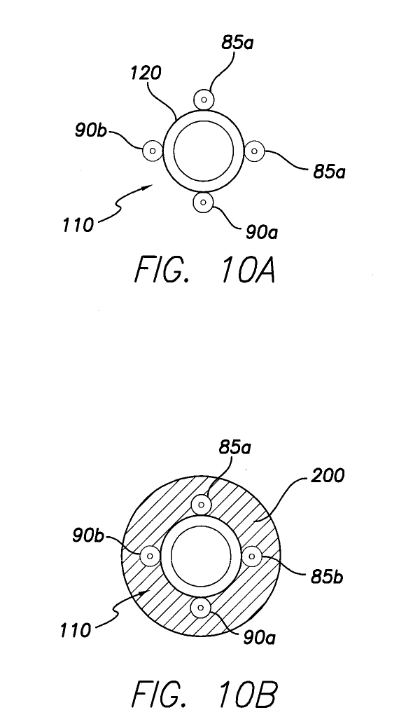

The embodiments described above with respect to any of FIGS. 5A-8C, discuss lead bodies 50 employing a helical core assembly 110 having a thin conformal jacket 125. However, the helical core assembly 110 may have other embodiments as indicated in FIGS. 10A and 10B, which are views similar to that depicted in FIG. 5C. For example, as depicted in FIG. 10A, in one embodiment, the helical core assembly 110 may first involve providing helically-routed conductors 85a, 85b, 90a, 90b in any number or arrangement about the liner tube 120. As can be understood from FIG. 10B, an infill material 200 may be provided about the combination of the inner tube 120 and the conductors 85, 90 to form an isodiametric helical core assembly 110. In one embodiment, the infill material 200 may be PTFE, ETFE, PEBAX, silicone rubber, polyurethane, SPC, etc. The infill material 200 may be provided about the combination of inner tube 100 and conductors 85, 90 via coextrusion, reflow or other methods. The isodiametric helical core assembly 110 may then be assembled into a lead body 50 as already described herein.

Although the present invention has been described with reference to preferred embodiments, persons skilled in the art will recognize that changes may be made in form and detail without departing from the spirit and scope of the invention.

What is claimed is:

1. An implantable medical lead comprising:
a longitudinally extending body including a distal end and a proximal end;
a helical core assembly extending between the distal and proximal ends; and
an outer jacket about the helical core assembly, wherein the helical core assembly includes at least one helical ridge.

2. The lead of claim 1, wherein the at least one helical ridge is at least two helical ridges and the helical core assembly further includes at least two helical troughs.

3. The lead of claim 2, wherein the at least two helical ridges define the at least two helical troughs.

4. The lead of claim 2, wherein the helical core assembly further comprises at least two electrical conductors radially spaced apart from each other about a central longitudinal axis of the lead body and helically extending about the central longitudinal axis.

5. The lead of claim 4, wherein each of the respective at least two helical ridges is defined in part by a respective one of the at least two electrical conductors.

6. The lead of claim 5, wherein the helical core assembly further comprises an inner tube liner about which the at least two electrical conductors helically extend.

7. The lead of claim 6, wherein the inner tube liner defines a central lumen of the lead body.

8. The lead of claim 6, wherein the helical core assembly further comprises a conformal jacket that is about the inner tube liner and the at least two electrical conductors, the conformal jacket generally conforming to the inner tube liner and the at least two electrical conductors.

9. The lead of claim 8, wherein the conformal jacket conforming to the at least two electrical conductors corresponds to the at least two helical ridges, and the conformal jacket conforming to the inner tube liner corresponds to the at least two helical troughs.

10. The lead of claim 2, wherein the outer jacket occupies at least a portion of the at least two troughs.

11. The lead of claim 2, further comprising a first electrical conductor helically routed along one of the at least two troughs.

12. The lead of claim 2, further comprising a mechanical element helically routed along one of the at least two troughs.

13. A method of assembling a medical lead, the method comprising:
providing a longitudinally extending helical core assembly including at least one helical ridge; and
providing an outer jacket about the helical core assembly.

14. The method of claim 13, wherein the at least one helical ridge is at least two helical ridges and the helical core assembly further includes at least two helical troughs.

15. The method of claim 14, wherein the helical core assembly further comprises:
at least two electrical conductors radially spaced apart from each other about a central longitudinal axis of the lead body and helically extending about the central longitudinal axis;
wherein each of the respective at least two helical ridges is defined in part by a respective one of the at least two electrical conductors.

16. The method of claim 15, wherein the helical core assembly further comprises:
an inner tube liner about which the at least two electrical conductors helically extend; and
a conformal jacket that is about the inner tube liner and the at least two electrical conductors, the conformal jacket generally conforming to the inner tube liner and the at least two electrical conductors.

17. The method of claim 16, wherein the outer jacket is caused to occupy at least a portion of the at least two troughs.

18. The method of claim 16, further comprising helically routing a first electrical conductor along one of the at least two troughs.

19. The method of claim 16, further comprising helically routing a mechanical element along one of the at least two troughs.

20. The method of claim 14, wherein the helical core assembly is provided as a prefabricated unit.

21. The method of claim 20, wherein the helical core assembly includes a removable core wire that serves as a mandrel until removed from the core assembly.

22. The method of claim 14, wherein the outer jacket is reflowed about the helical core assembly.

23. An implantable medical lead comprising:
a longitudinally extending body including a distal end and a proximal end; and
a helical core assembly extending between the distal and proximal ends;
wherein the helical core assembly includes an inner tube liner and a helically-routed conductor having a wind pitch of between approximately 0.05″ and approximately 0.3″ and routed about the inner tube liner.

24. The lead of claim 23, wherein an infill polymer material extends around the helical core assembly to cause the helical core assembly to be generally isodiametric.

25. The lead of claim 24, further comprising an insulation layer extending around the infill polymer material.

26. The lead of claim 25, further comprising an outer jacket extending around the insulation layer.

27. The lead of claim 23, wherein the at least one helically-routed conductor forms at least one helical ridge.

28. The lead of claim 27, wherein the at least one helical ridge is at least two helical ridges and the helical core assembly further includes at least two helical troughs.

29. The lead of claim 28, wherein the at least two helical ridges define the at least two helical troughs.

30. The lead of claim 27, wherein the helical core assembly further comprises a conformal jacket that is about the inner tube liner and the at least one electrical conductor, the conformal jacket generally conforming to the inner tube liner and the at least one electrical conductor.
